(19) United States
(12) Patent Application Publication
(10) Pub. No.: US 2019/0117362 A1
(43) Pub. Date: Apr. 25, 2019

(54) TISSUE PRODUCTS WITH VARIATIONS IN MECHANICAL PROPERTIES AND METHODS OF TREATMENT

(71) Applicant: LifeCell Corporation, Madison, NJ (US)

(72) Inventor: William A. Daunch, Cary, NC (US)

(21) Appl. No.: 16/165,299

(22) Filed: Oct. 19, 2018

Related U.S. Application Data
(60) Provisional application No. 62/599,539, filed on Dec. 15, 2017.
(60) Provisional application No. 62/575,063, filed on Oct. 20, 2017.

(51) Int. Cl. A61F 2/00; A61L 27/36

(52) U.S. Cl. CPC A61F 2/0059; A61L 27/362; A61F 2250/0024; A61F 2002/0081; A61F 2220/0016; A61L 27/3641

(57) ABSTRACT

Devices with improved biomechanical properties and methods for treating tissue are provided. The compositions may comprise acellular tissue matrices specifically shaped and sized for facial and neck implantation and having variations in mechanical and/or biological properties. Also provided are methods for treating tissue using these devices.

The present disclosure claims priority under 35 USC § 119 to U.S. Provisional Application No. 62/575,063, which was filed on Oct. 20, 2017, and to U.S. Provisional Application No. 62/599,539, which was filed on Dec. 15, 2017, both of which are incorporated herein by reference in their entirety.

The present disclosure relates to tissue treatment devices with improved biomechanical properties, including tissue products to support facial structures. The devices can include acellular tissue matrices (ATMs) specifically shaped and sized for facial implantation and having variations in mechanical or biological properties.

The use of acellular tissue matrices, including acellular dermal matrices (ADMs), in surgical procedures has become increasingly popular with plastic surgeons. Such materials provide a number of advantages and can be used to replace or augment supportive structures after, for example, facial reconstruction, mastectomy, breast augmentation, abdominal reconstruction, or any other suitable surgical procedure that may require additional structural support. Such tissue products can also be useful in aesthetic procedures (e.g., facelift surgery, neck lift surgery) by providing additional support and providing a biologic material that becomes resorbed and remodeled. However, these tissue support materials (e.g., ADMs) can be monolithic in design—having mechanical properties and dimensions that are essentially consistent throughout. The devices can limit the surgeon's, or other suitable practitioner's, ability to create a more natural post-surgical appearance when implanted. Additionally, some tissue support devices may include one or more meshes, sutures, and/or barbed sutures that are also monolithic in design. These meshes, sutures, and/or barbed sutures may be associated with a particular compliance (e.g., stiffness, elasticity, etc.) that may not be suitable for the compliance requirements of any native tissue at the implantation site.

In addition, surgeons may be required to perform excessive customization in and around the tissue support implant site to reduce patient discomfort and/or pain, or to achieve a particular aesthetic result. A surgeon may be limited in selection of tissue products that provide a post-surgical appearance that optimally contours the appropriate anatomical features of a patient's body (e.g., face, abdomen, etc.). Therefore, there is currently a need for tissue support devices having modified mechanical properties to match the anatomy and heterogeneous biomechanics of the native tissue (e.g., facial, abdominal, etc.) or more appropriately provide desired post-surgical mechanics.

There is currently a need for improved tissue support matrices (e.g., acellular dermal matrices) with variable mechanical properties, predictable fixation, or variable biological properties in order to more effectively treat appropriate anatomical structures of a patient's body (e.g., face, neck, abdomen, etc.). For example, to treat various facial features (e.g., lines, wrinkles, insufficient volume, or less than desirable shapes or forms), improved tissue matrices, such as ADMs, may be used. These materials may serve as tissue support devices that may conform to the appropriate anatomical structure of a face to provide improved support (e.g., to accommodate animation of the face or provide a more natural facial appearance, aesthetic outcome, or facial expression).

Additionally, there is currently a need for improved tissue support matrices (e.g., acellular dermal matrices) with variable mechanical properties, predictable fixation, or variable biological properties in order to more effectively treat and address signs of aging and/or improve the aesthetic appearance in the neck and jawline area of a patient. One particular procedure used to improve the aesthetic appearance of the neck area is a neck lift, which can address excess fat and skin relaxation in the lower face and chin areas, remove loose neck skin, or reduce the appearance of muscle banding, such as platysmal banding, in the neck. For treatment of muscle banding especially, such as platysmal banding, increased support for the platysma muscle can counteract natural loosening of the fascia attachments surrounding the muscle to reduce the appearance of bands and/or sagging in the neck area. To improve neck lift outcomes, improved tissue matrices, such as ADMs, may also be used.

The present application provides improved tissue support devices including tissue matrix materials shaped and/or sized to improve implantation during surgical procedures (e.g., facial, neck or abdominal reconstruction). The tissue matrix devices may be mechanically and/or biologically modified to provide varying regions of stretch and elasticity, wherein the varying regions may include one or more localized fenestrations or openings (e.g., perforations, indentations, slits, holes, and/or dimples, etc.). These openings may be selected to have a size, shape, number, spacing and/or orientation to provide desired material mechanics. Moreover, the tissue matrices may be alternatively or additionally modified by treating the tissue(s) with enzymes and/or chemicals to either soften the tissue or to cross-link the tissue to permit the tissue to increase in stiffness.

Also provided are methods of treatment that include implanting the disclosed devices within anatomical structures (e.g., within or around the face or other cranio-facial structures or the neck).

It will be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary embodiments of the present disclosure and, together with the description, serve to explain the principles of the disclosure.

FIG. 1A is a side perspective view illustrating an exemplary facial tissue support device near an exemplary treatment site according to aspects of the present disclosure;

FIG. 1B is an enlarged view of the facial tissue support of FIG. 1A;

FIG. 2A illustrates a lateral view of a distribution of relaxed skin tension lines on a patient;

FIG. 2B illustrates a frontal view of a distribution of relaxed skin tension lines on a patient;

FIG. 3A illustrates a configuration for facial tissue support devices implanted in a patient according to aspects of the present disclosure;

FIG. 3B illustrates a configuration for facial tissue support devices implanted in a patient according to aspects of the present disclosure;

FIG. 3C illustrates a configuration for facial tissue support devices implanted in a patient according to aspects of the present disclosure;

FIG. 3D illustrates a configuration for facial tissue support devices implanted in a patient according to aspects of the present disclosure;

FIGS. 4A-D illustrate a three-dimensional computational simulation of facial mapping of a patient, including the tissue support device in FIG. 4D, according to aspects of the present disclosure;

FIG. 5A illustrates an exemplary embodiment of a facial tissue support as implanted in a patient's face produced according to aspects of the present disclosure;

FIG. 5B illustrates an enlarged view of the device of FIG. 5A;

FIG. 6 illustrates an exemplary embodiment of a facial tissue support implanted in a patient's face according to aspects of the present disclosure;

FIG. 7A illustrates an exemplary embodiment of a facial tissue support without openings, according to aspects of the present disclosure;

FIGS. 7B-7H illustrate exemplary embodiments of facial tissue supports with various opening (e.g., perforation) patterns according to aspects of the present disclosure;

FIG. 8 illustrates an exemplary embodiment of a facial tissue support according to aspects of the present disclosure;

FIG. 9 illustrates an exemplary embodiment of a neck tissue support according to aspects of the present disclosure;

FIG. 10 illustrates the neck tissue support shown in FIG. 9 after modification by a user;

FIG. 11 illustrates the neck tissue support shown in FIG. 10 attached to a face of a patient; and

FIG. 12 illustrates the neck tissue support shown in FIGS. 10-11 attached to the face of the patient during significant movement of the patient's face and neck area.

### DETAILED DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any range described herein will be understood to include the endpoints and all values between the endpoints.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

As used herein “tissue product” will refer to any human or animal tissue that contains an extracellular matrix protein. “Tissue products” may include acellular or partially decellularized tissue matrices, as well as decellularized tissue matrices that have been repopulated with exogenous cells.

As used herein, the term “acellular tissue matrix” refers to an extracellular matrix derived from human or animal tissue, wherein the matrix retains a substantial amount of natural collagen and glycoproteins needed to serve as a scaffold to support tissue regeneration. “Acellular tissue matrices” are different from purified collagen materials, such as acid-extracted purified collagen, which are substantially void of other matrix proteins and do not retain the natural micro-structural features of the tissue matrix due to the purification processes. Although referred to as “acellular tissue matrices,” it will be appreciated that such tissue matrices may be combined with exogenous cells, including, for example, stem cells, or cells from a patient in whom the “acellular tissue matrices” may be implanted. Tissue matrices can be determined to be acellular or decellularized by using light microscopy to verify the absence of cells.

Various human and animal tissues may be used to produce products for treating patients. For example, various tissue products for regeneration, repair, augmentation, reinforcement, and/or treatment of human tissues that have been damaged or lost due to various diseases and/or structural damage (e.g., from trauma, surgery, atrophy, and/or long-term wear and degeneration) have been produced. Such products may include, for example, acellular tissue matrices, tissue allografts or xenografts, and/or reconstituted tissues (i.e., at least partially decellularized tissues that have been seeded with cells to produce viable materials).

A variety of tissue products have been produced for treating soft and hard tissues. For example, ALLODERM® and STRATTICE™ (LIFECELL CORPORATION, BRANCHBURG, N.J.) are two dermal acellular tissue matrices made from human and porcine dermis, respectively. Although such materials are very useful for treating certain types of conditions, materials having different biological and mechanical properties may be desirable for certain applications. For example, ALLODERM® and STRATTICE™ have been used to assist in the treatment of structural defects and/or to provide support to tissues (e.g., for abdominal walls or in breast reconstruction), and their strength and biological properties make them well suited for such uses. However, modifying those materials to include variations in mechanical or biological properties (e.g., by adding openings, perforations, or fenestrations in predefined patterns, and/or by chemically or enzymatically modifying the materials) may further improve the materials' uses. Further, modifying the devices to include preformed shapes and sizes for various applications can be useful.

Surgeons, or any other suitable medical practitioner, may seek adaptable product solutions for variable facial laxity in patients. These solutions may be adjusted and tuned so as to be customizable for maximum effect for a particular procedure (e.g., facial rejuvenation, eyebrow lift, eyelid surgery, facelift surgery, neck lift surgery, abdominal surgery, or any other suitable surgery relating to muscular movements). In some examples, tissue support devices according to aspects of the present disclosure may be use for facial aesthetic solutions. Facial aesthetic solutions may require more delicate or customized reconstruction and support, for example, in procedures involving brow lifts, face lifts, and the treatment of the neck or other cranio-facial structures. In these procedures, improved heterogeneous, regionally compliance-matched fixation properties and predictable integration may be desired.

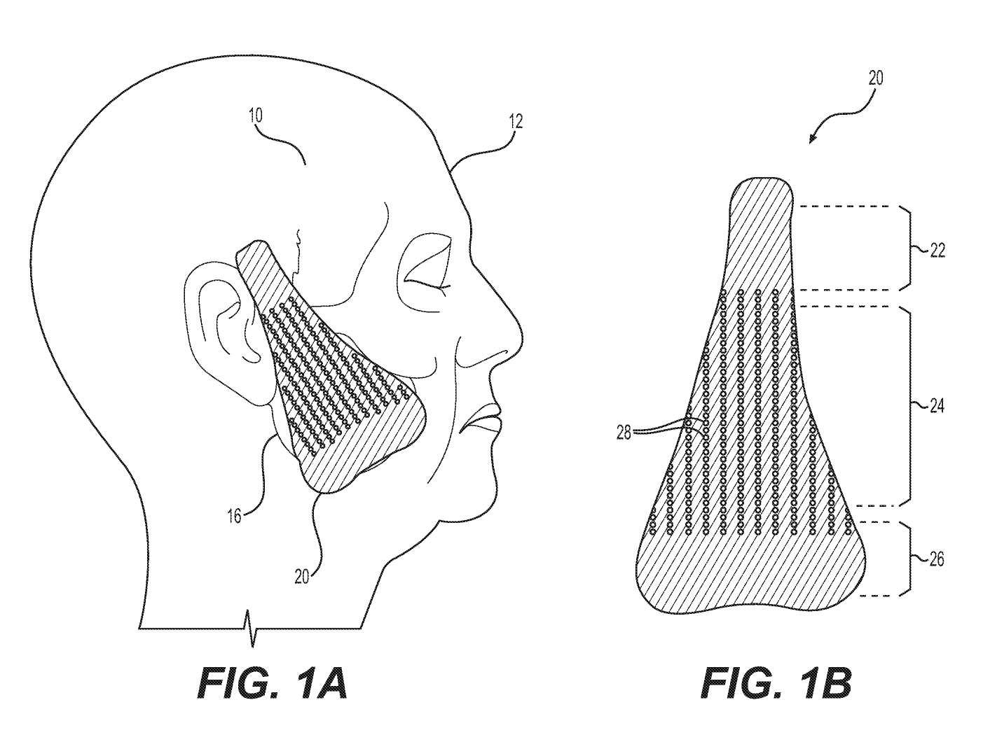

With initial reference to FIGS. 1A and 1B, an exemplary tissue support device 20 is shown. Tissue support device 20 may comprise an acellular tissue matrix, such as an acellular dermal matrix with variable mechanical and/or biological properties according to aspects of the present disclosure. As shown in FIG. 1A, tissue support device 20 may be implanted within a selected area 16 of a face 12 of a patient 10. FIG. 1A illustrates a simple design for a device 20 that may be used for a mid-face lift (or brow lift).

Although discussed particularly with reference to acellular dermal matrices such as ALLODERM® or STRATTICE™, or similar materials, the devices discussed herein can be produced from a number of suitable acellular tissue matrices known in the regenerative medicine and surgical fields including those produced, for example, from small intestine, small intestine submucosa, other gastrointestinal layers (e.g., parts of the stomach), bladder or layers of bladder, or other known acellular tissue matrices. For example, a number of biological scaffold materials are described in Badylak, et al., “Extracellular Matrix as a Biological Scaffold Material: Structure and Function,” Acta Biomaterialia (2008), doi:10.1016/j.actbio.2008.09.013 (the entire contents of which is incorporated herein by reference), and the devices discuss herein could be produced with such material.

With continuing reference to FIG. 1B, tissue support device 20 may comprise a number of sections, including a first anchoring section 22, a middle section 24, and a second anchoring section 26. First and second anchoring sections 22 and 26 may be used to surgically attach tissue support device 20 to native facial tissue of patient 10. First and second anchoring sections 22 and 26 may comprise suture areas or areas for other suitable surgical attachments, such as, for example, barbed sutures or meshes. Further, in some cases, a surgeon may elect to secure parts of section 24 to tissues.

Dimensions and/or sizes of sections 22, 24, and 26 of tissue support device 20 may change as a function of the appropriate anatomical site of the face of the patient (e.g., distance between the incision point near a patient's ear and the lower undermined cheekbone area, etc.). The amount of material in the first and second anchoring sections 22 and 26 may change as additional, or fewer, suture bites, anchoring points, and/or other surgical fixations may be required. In one embodiment, middle section 24 may comprise about 80% of a dimension (e.g., length, width, etc.) of tissue support device 20, while first and second anchoring sections 22 and 26 may comprise the remaining about 20%. In some embodiments, middle section 24 may comprise about 70%, while first and second anchoring sections 22 and 26 may comprise the remaining about 30%.

Middle section 24 may include a modified zone, as shown in FIG. 1B. In an exemplary embodiment, the modified zone may provide for biomechanical properties that are more desirable for treatment of a selected anatomic site of a patient 10. As shown in FIG. 1B, a group of openings 28 may be disposed along the modified zone of middle section 24 of tissue support device 20. The modified zone may be disposed along either a portion or all of modified middle section 24 (e.g., a portion of or the entire width/length of section 24). Further, variations in the shape, size, and configuration are contemplated depending on the desired use.

In an exemplary embodiment, openings 28 may comprise perforations, indentations, slits, holes, dimples, and/or any other suitable openings to improve gradient mechanical properties of tissue support device 20. In some embodiments of the present disclosure, tissue support device 20 may comprise varying regions that may include one or more localized openings. These openings may vary in size, shape, number, spacing and/or orientation relative to tissue support device 20. In an exemplary embodiment, openings 28 may be used to alter the mechanical properties selectively across the ATM of tissue support device 20.

According to several aspects of the present disclosure, ATMs may be formed of a specific shape and/or size for facial and/or neck implantation and may have variations in mechanical properties selected based on anatomical structures and properties of the face 12 of the patient 10. In doing so, tissue support device 20 may provide better support, conformability, more natural feel, or overall better handling. As such, the devices disclosed herein can provide a more natural post-surgical appearance or expression or otherwise provide better surgical results.

As discussed above, the devices can include modified zones with openings. But, as mentioned previously, the modified zones may be created by alternatively or additionally treating the tissue support device 20 with enzymes and/or chemicals to soften the tissue or to cross-link the tissue to increase the tissue's stiffness or affect other properties.

In some embodiments, modified zones may be provided with a chemical or enzymatic treatment, e.g., as described in U.S. Pat. No. 9,592,254 B2, issued Mar. 14, 2017, and assigned to LIFECELL CORPORATION of BRANCHBURG, N.J., the entirety of which is herein incorporated by reference.

Further, although the formation of openings or treatment with chemicals or enzymes is described with respect to middle section 24, it may be desirable to provide localized cross-linking and/or enzymatic treatment to parts of the first and second sections 22, 26. For example, first and second sections 22, 26 may be treated to increase strength (e.g., suture retention strength). It can be appreciated that to achieve varying mechanical and/or biological properties, tissue support device 20 may be chemically treated in only one region, all regions, or select regions, as determined by a surgeon, or other suitable practitioner, or design engineers.

The present disclosure also provides methods for treating tissues to provide variable mechanical and/or biological properties. According to various embodiments, a method for treating a tissue matrix is provided. The method may comprise selecting a collagen-containing tissue matrix and cross-linking or enzymatically treating select portions of the tissue matrix (e.g., regions 22, 24, and/or 26 of tissue support device 20) to produce a tissue matrix having mechanical and/or biological properties that vary across the tissue matrix. In some embodiments, the tissue matrix may include an acellular tissue matrix according to aspects of the present disclosure. In certain embodiments, the tissue matrix may comprise a dermal tissue matrix.

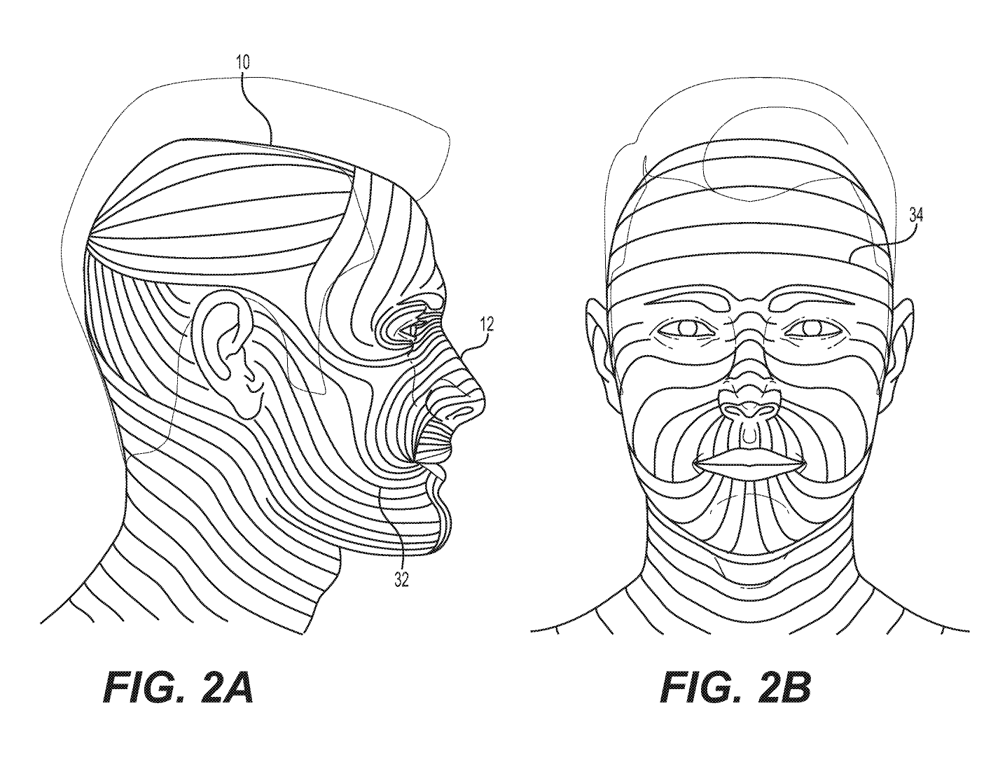

FIGS. 2A and 2B illustrates a distribution of relaxed skin tension lines 32 (lateral view in FIG. 2A) and 34 (anterior view in FIG. 2B) of face 12 of patient 10. These lines are also known as Kraissl's lines. When observing live patients, Kraissl's lines may demonstrate the benefits of surgical incisions following the plane of tissue folds on a face of a patient to minimize scarring. When tissue support device 20 is implanted within a portion of a face 12 of patient 10, Kraissl lines 32, 34 may assist a surgeon in demonstrating where skin creases (in live patients) form. These skin creases may form perpendicular to the direction of muscle pull in a patient's face. Moreover, Langer lines (not shown) may be used to assess regional collagen orientation to provide the surgeon with ideal incision points on face 12 of patient 10. Moreover, lines 32 and 34 may assist in guiding a surgeon in planning elective incisions for improved aesthetic facelift solutions. Further, these lines can be used as guides for positioning the device against the local muscle tissue and may be used to identify areas of local deficits that need to be addressed. They are valuable in allowing the surgeon to select the gradient structure that will be required to overcome the laxity or deficit. Such lines may be used for selection of an appropriate device as discussed herein or to aid in production of customized devices. It should be appreciated that, while lines of the face are shown in FIGS. 2A-B, lines in the neck area may also be utilized to select an appropriate gradient structure for a tissue support device for the neck area as will be described further herein.

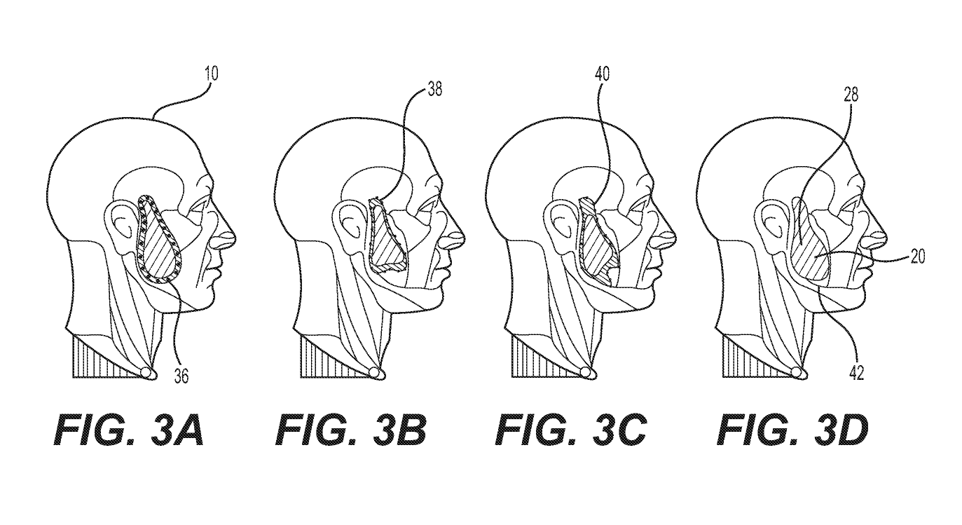

FIGS. 3A-D illustrate an exemplary simulation using three-dimensional (“3D”) dynamic biomechanical simulation of the facial anatomy of patient 10. Simulations may be performed by one or more processors. Data generated by the one or more processors may be stored locally or on an external off-site storage facility. Data may be collected over a single simulation or multiple simulations to map the facial anatomy of patient 10 to generate a tissue support best suited for the anatomical and physiological parameters of the patient's face (or other suitable body part involving muscular movement).

The computational simulation, as shown in FIGS. 3A-D, illustrates Langer/Kraissl line “tension maps” 36, 38, 40, and 42. Tension maps 36, 38, 40, and 42 may be used by a surgeon, or any other suitable practitioner, as a guide for designing new or customized devices. For example, based on tension maps 36, 38, 40, and 42, tissue support device 20 may be implanted within patient 10 to provide improved aesthetic facelift solutions such as, for example, to aid in the design of fit-for-purpose facelift solutions that are more customized to natural anatomical variations of a patient's face.

In an exemplary embodiment, these three-dimensional biomechanical simulations may guide formation of a pattern of openings 28 on tissue support device 20, as shown in FIG. 3D, or guide other modifications such as enzymatic or chemical modification. For example, openings 28 may be formed in a pattern that dictates the amount of deflection of a particular area or that produces a desired flexibility (e.g., tensile modulus or pliability). In some examples, openings 28 may be formed on tissue support device 20 to address potential areas where stress may develop. In another example, openings 28 may be formed to address concerns relating to differential strain or differential stress on various regions of tissue support device 20. Dynamic 3D biomechanical modeling may assist in the foregoing.

It is contemplated that the devices disclosed herein can be prefabricated with shapes and properties preselected for a variety of types of procedures (e.g., with a size, shape, and mechanical properties selected for common patient characteristics including face size and structure, tissue mechanics, or other surgical factors). Furthermore, the devices may be custom fabricated for a particular patient, and as such, the mechanical modelling discussed herein provides a method for developing customized implants.

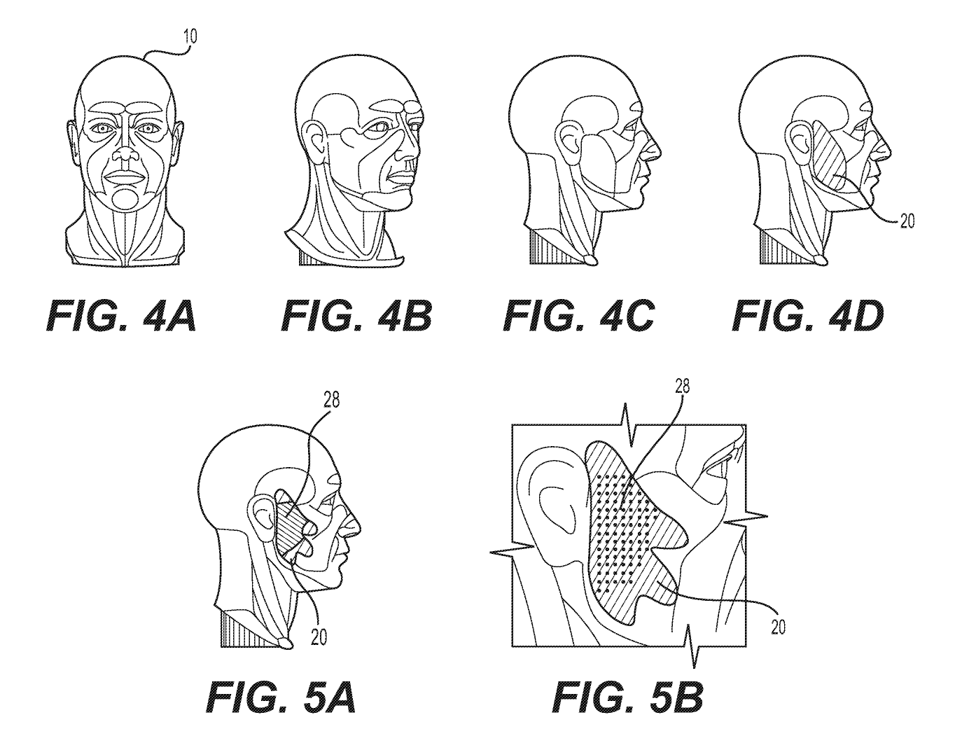

FIGS. 4A-D illustrates an exemplary embodiment of a progressive design concept iteration according to aspects of the present disclosure. As shown, FIG. 4A-D demonstrates further 3D facial mapping to assist a surgeon, or other suitable practitioner, in planning elective incisions for improved aesthetic facelift solutions.

With reference now to FIGS. 5A and B, as shown is an exemplary tissue support device 20′ having varying regions, including openings 28. As shown, openings 28 may be designed in any kind of pattern to cover a specific area of tissue support section 24. Openings 28 may include specific shapes and/or specific directions. Once a specific and unique opening pattern has been formed on tissue support section 24 of a tissue support, the tissue support may then undergo 3D biomechanical simulations, as alluded to earlier in this disclosure, to obtain a measurable performance response. Once the measurable performance response is received, a surgeon, or other suitable practitioner, may then match the performance response of the openings to the compliance expectations of a particular anatomical situation.

Moreover, as shown in FIG. 5B, tissue support device 20′ may be comprised or formed from an ATM that may be anchored to one or more muscle groups or other tissues found near or within a patient's face. For example, as shown, tissue support device 20′ may be striated in a “leaf-like” pattern. Moreover, openings 28 may be formed in one or more regions of tissue support device 20′, as shown. Openings 28 may be inserted in tissue support device 20′ to incorporate the complexity and heterogeneity of facial biomechanics. In an example, tissue product 20′ may significantly improve the standard of care in face lift procedures. These tissue products 20′ may result in longer lasting lifts, more predictable aesthetic outcomes, and/or more effective tissue integration. Improved tissue integration may yield reduced irritation, healing time, and/or scarring in a patient.

In an exemplary embodiment, a user (e.g., a surgeon or other suitable medical practitioner) may select an opening pattern of 40×40 holes (or some range of holes from 10-100, or more for some patterns) arranged parallel to the direction of the tissue support's elongation. Once the user has selected a specific pattern, the user may simulate, via 3D biomechanical dynamic modeling, the tissue support's mechanical properties to receive a measurable performance response. Once the feedback is acquired, the tissue support may be further manipulated, by additional openings in the tissue support, additional mechanical manipulation of the tissue support, chemical manipulation of the tissue support (e.g., cross-linking, etc.), and/or additional fixation points used by the surgeon during implantation, to match the compliance expectations of a particular anatomical situation so that the tissue support conforms to the appropriate anatomical structures of the face to provide better and/or improved support. In doing so, the tissue support, when implanted in the patient's face, may accommodate animation of the face, provide a more natural appearance/expression, reduce irritation at the site of the implant, reduce pain, etc.

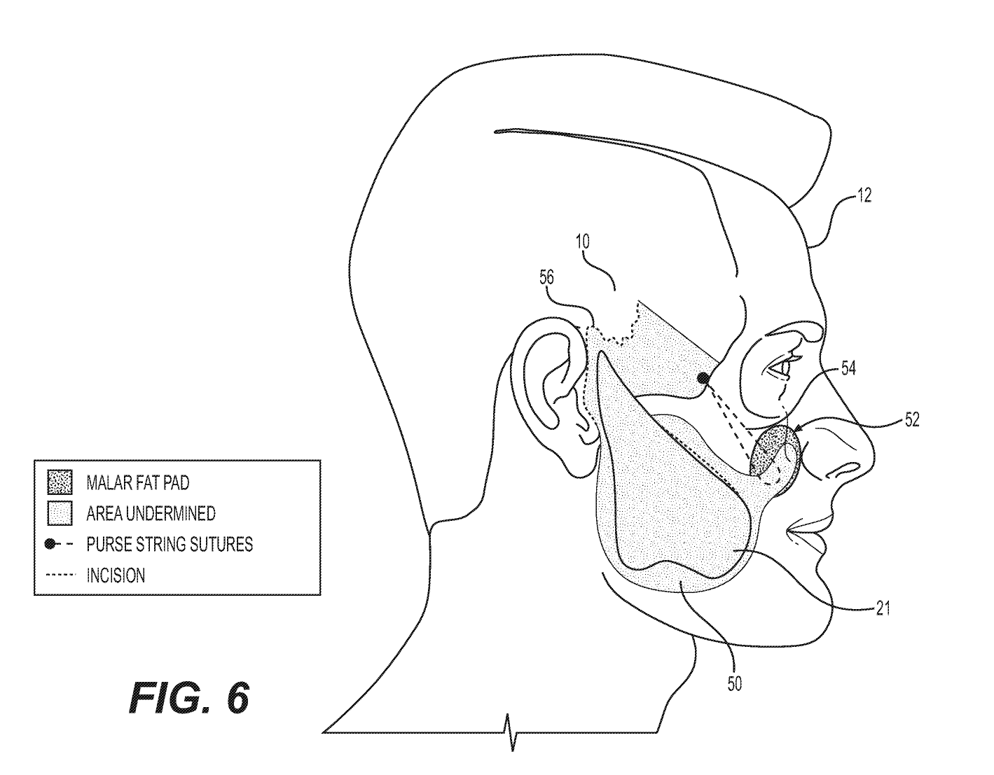

FIG. 6 is illustrative of an exemplary tissue support device 21 inserted within a facial region of patient 10 according to aspects of the present disclosure previously described. As shown, tissue support device 21 is implanted over an undermined facial area 50 and between an incision site 56 and Malar fat pad 52. For illustrative purposes, a purse string suture 54 connecting two connective tissue portions of undermined area 50 is shown to exemplify the complexity and customization required to make traditional surgical solutions work. Exemplary tissue support device 21, however, may permit a surgeon, or other suitable medical practitioner, to create facelift solutions with improved gradient mechanical properties.

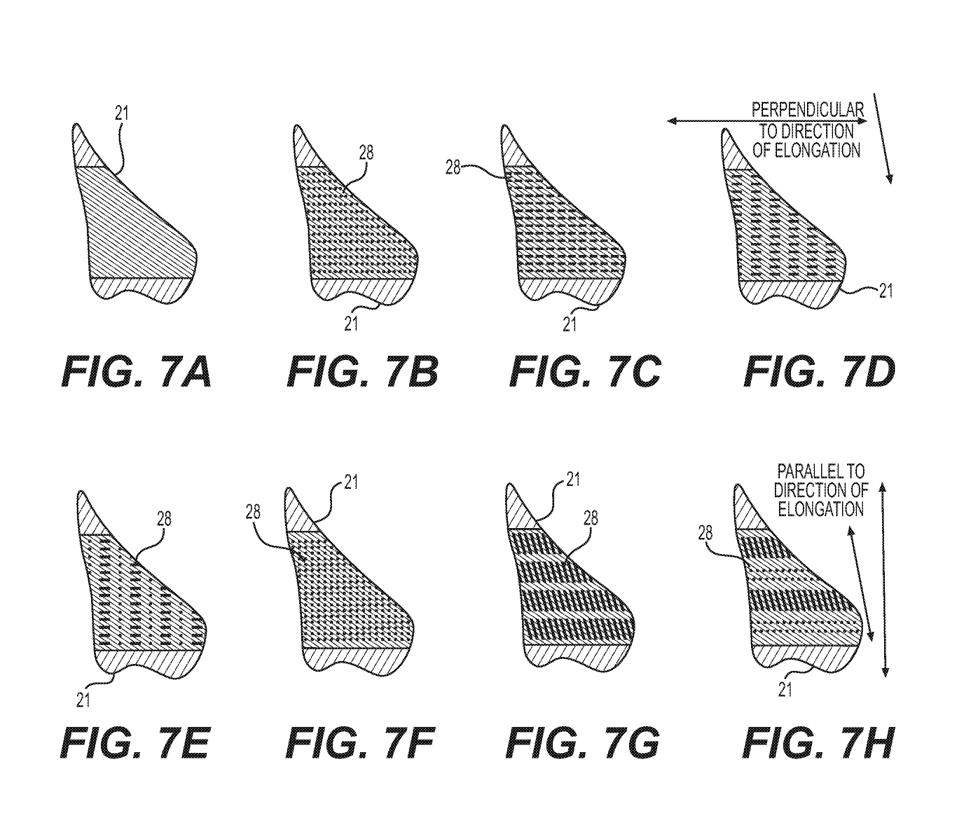

FIGS. 7B-7H are exemplary embodiments of tissue support device 21, with various opening designs or patterns, according to aspects of the present disclosure. FIG. 7A is an exemplary embodiment of tissue support device 21 having no openings. FIGS. 7B-7H, for example, show a tissue support product 21 having openings 28 disposed along a modified zone of middle section 24. As shown in FIGS. 7C-7E, openings 28 may be arranged perpendicular to the direction of elongation of tissue support device 20. Moreover, openings 28 may also be arranged parallel to the direction of elongation of tissue support device 21 as shown in FIGS. 7F-7H. In these examples, openings 28 arranged parallel to the direction of elongation may be stiffer and may elongate, but may not elongate to the extent openings 28 arranged perpendicular to the direction of elongation will.

It can be appreciated that openings 28, in other exemplary embodiments, may include alternating patterns of perpendicular and/or parallel openings. In some embodiments, openings 28 may be the same pattern (e.g., homogenous) or a combination/permutation of patterns (e.g., heterogeneous). Patterns may include: apertures, slits, indentations, grooves, slots, pockets, recesses, dimples, and/or holes in various shapes (e.g., triangular, rectangular, octagonal, square, rhombus, trapezoidal, etc.). In an exemplary embodiment, combinations and/or permutations of openings 28 may be incorporated into the design of tissue support device 21 to manipulate the gradient mechanical properties of the implantable tissue product.

For example, slits may be incorporated parallel to the direction of elongation or may be incorporated perpendicular (or angled with respect to) to the direction of elongation depending on what section of tissue support device 21 may require elongation. For example, if more elongation in the lower 30% of tissue support device 21 is preferred, then less elongation in the upper 30% may be desired. As described throughout the present disclosure, openings 28 may be used to achieve these gradient mechanical properties. Moreover, 3D dynamic biomechanical simulation, as described earlier, may also be used to create an optimal opening pattern for tissue support device 21.

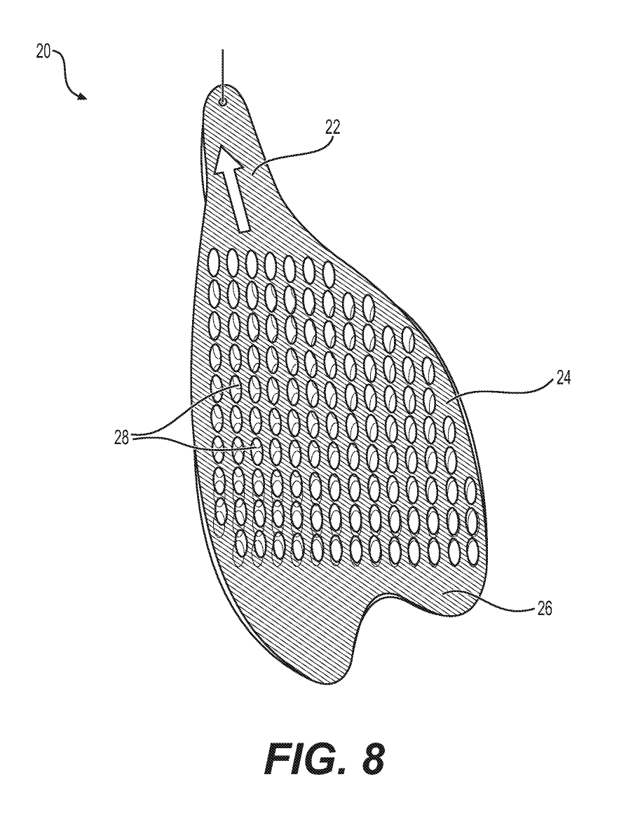

Referring now to FIG. 8, an illustrative embodiment of an exemplary tissue support device 20 according to aspects of the present disclosure is shown. Tissue support device 20 may comprise a first anchoring region 22, a middle region 24, and a second anchoring region 26. First and second anchoring regions 22 and 26 may be used to surgically attach tissue support device 20 to native facial tissue of patient 10 (e.g., cranio-facial muscles, fascia, or other appropriate tissue). Surgical fixation may be performed with attachment elements including, but not limited to, sutures, barbed sutures, tacks, meshes, clips, biologic adhesives, or any other suitable technique used by a surgeon or practitioner to insert and fixate an acellular dermal tissue matrix to native tissue.

As shown in FIG. 8, in an exemplary embodiment, a group of openings 28 may be disposed along the modified zone of middle region 24 of tissue support device 20. In this example, the openings 28 have an elongated direction parallel to the direction of elongation of tissue support device 20. Openings 28 are circular or ovoid in shape, as shown; however, openings 28 may retain varying shapes and have uniform or non-uniform patterns (e.g., continuous circular holes, circle-square-circle-square, triangle-circle-triangle, etc.)

As shown in FIGS. 7A-7H and 8, changing the opening type may be desirable to achieve a specific biomechanical response. For example, openings 28 may be arranged parallel or perpendicular to the direction of ordered collagen and/or disordered collagen found in a patient's face; in other exemplary embodiments, openings of the tissue support may also be arranged parallel or perpendicular to the direction of ordered collagen and/or disordered collagen found in a patient's neck. Human tissue naturally has varying degrees of both ordered and unordered collagen, and, consequently, openings 28 may be manipulated or designed to accommodate these collagen arrangements. In some embodiments, openings 28 may be arranged perpendicular to the direction of collagen present in a patient's face and/or neck area. Yet, in other embodiments, combinations or permutations of openings 28 (e.g., various shapes and/or sizes of holes, indentations, slits, etc.) may be selected to accommodate various types of collagen (e.g., to accommodate the different types of anatomical structures of the face, the abdomen, the breasts, or any other portion of the body requiring a muscle movement, etc.). Depending on whether the collagen is ordered or disordered or somewhere in between—the direction of openings 28 (e.g., indentations, dimples, etc.) may be changed to achieve specific gradient mechanical properties. For example, openings 28 may be oriented with the collagen, oriented against the collagen, or may be positioned in some orientation in a non-oriented structure. In doing so, a surgeon, or other suitable practitioner, may create customizable angles in tissue support device 20 to create a desired mechanical response. In some embodiments, 3D dynamic biomechanical simulations may assist in the foregoing process.

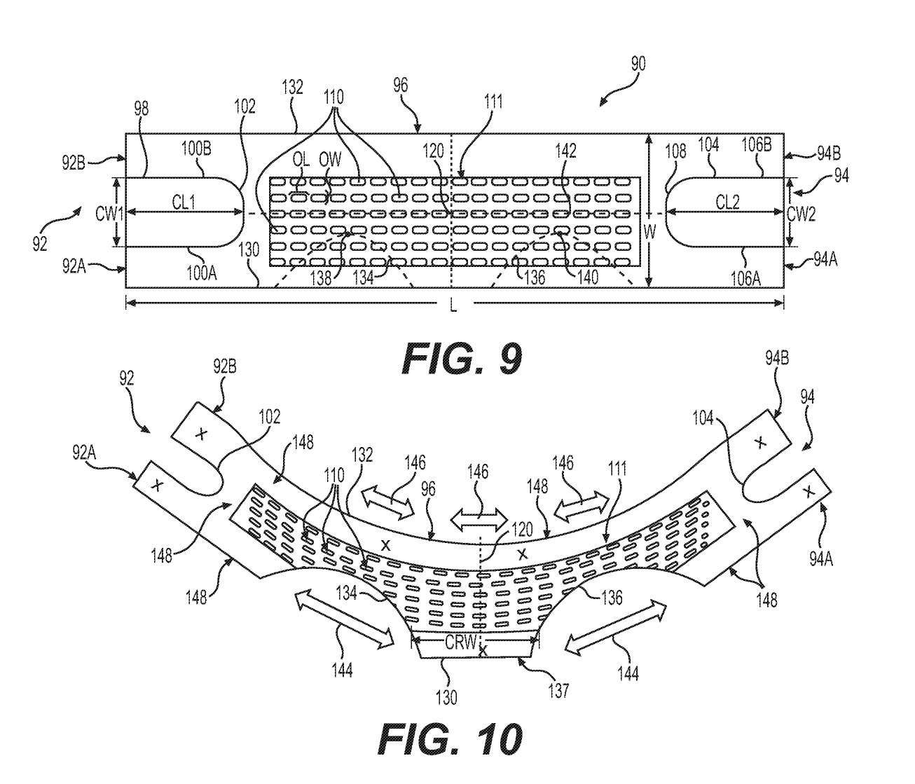

Referring now to FIG. 9, another exemplary embodiment of a tissue support 90 formed in accordance with the present invention is shown. Similar to tissue support 20 (or 21), the tissue support 90 comprises a sheet of acellular tissue matrix, such as porcine dermal matrix, and has a first end section 92, a second end section 94, and a middle section 96 connecting the first end section 92 and second end section 94. The first end section 92 and the second end section 94 may be configured as anchoring sections that are disposed on opposite ends of the tissue support 90 and are suitable to anchor the tissue support 90 to, for example, facial and/or neck tissue of a patient, as will be described further herein. As shown in FIG. 9, the tissue support 90 is formed generally in the shape of a rectangular sheet having various portions removed, as will be described further herein, but it should be appreciated that the tissue support 90 may be formed in any desired shape.

In some exemplary embodiments, the first end section 92 may include a first zygomatic attachment flap 92A and a first mastoid attachment flap 92B that are separated from one another by a first cutout 98 formed in the first end section 92. It should be appreciated that while the flaps 92A, 92B are previously referred to as a “zygomatic attachment flap” and a “mastoid attachment flap,” respectively, the attachment flaps 92A, 92B may be suitably sized and configured for attachment to any desired tissue structure and/or region. In some exemplary embodiments, the first cutout 98 may be formed so the first attachment flaps 92A, 92B both define a respective inner straight edge 100A, 100B that meets a curved edge 102 formed in the tissue support 90. In some exemplary embodiments, the first cutout 98 may be formed with a first cutout length CL1 that is approximately 10-30% of a total length L of the tissue support 90; in some exemplary embodiments, the cutout 98 may be formed with a first cutout width CW1 that is approximately 40-50% of a total width W of the tissue support 90. It should be appreciated that the overall shape and dimensions of the first end section 92 shown in FIG. 9 is exemplary only, and the shape and dimensions of the first end section 92 may be varied, as desired, to accommodate different anchoring elements and/or attach to various attachment sites. Further, all or a portion of the first end section 92 may be chemically and/or enzymatically treated to, for example, strengthen the first end section 92 for attachment, as previously described.

Similarly, in some exemplary embodiments, the second end section 94 may include a second zygomatic attachment flap 94A and a second mastoid attachment flap 94B that are separated from one another by a second cutout 104 formed in the second end section 94. It should be appreciated that while the second attachment flaps 94A, 94B are previously referred to as a “zygomatic attachment flap” and a “mastoid attachment flap,” respectively, the attachment flaps 94A, 94B may be suitably sized and configured for attachment to any desired tissue structure and/or region. In some exemplary embodiments, the second cutout 104 may be formed so the second attachment flaps 94A, 94B both define a respective inner straight edge 106A, 106B that meets a curved edge 108 formed in the tissue support 90. In some exemplary embodiments, the second cutout 104 may be formed with a second cutout length CL2 that is approximately 10-30% of the total length L of the tissue support 90; in some exemplary embodiments, the cutout 104 may be formed with a second cutout width CW2 that is approximately 40-50% of the total width W of the tissue support 90. It should be appreciated that the overall shape and dimensions of the second end section 94 shown in FIG. 9 is exemplary only, and the shape and dimensions of the first end section 92 may be varied, as desired, to accommodate different anchoring elements and/or attach to various attachment sites. Further, all or a portion of the second end section 94 may be chemically and/or enzymatically treated to, for example, strengthen the first end section 94 for attachment, as previously described.

To allow for customization by a user, such as a trained surgeon or other medical professional, during, for example, a neck lift surgery, the tissue support 90 may be formed to have a total length L greater than a total distance between two desired tissue attachment sites of the patient, such as a distance between the ears of a patient along the mandible, to allow the user to trim off part of the first end section 92 and/or the second end section 94 to reduce the total length L to the desired length during a procedure. In some exemplary embodiments, the total length L of the tissue support 90 may be between 20 cm and 35 cm, but the tissue support 90 may also be formed with a larger or smaller total length L depending on the surgical application. In some exemplary embodiments, the total width W of the tissue support 90 may be between 0.5 cm and 8 cm, but the tissue support 90 may also be formed with a larger or smaller total width W, depending on the surgical application. It should be appreciated that the total width W of the tissue support 90 may also be chosen so a user can trim off portions of the tissue support 90 to a desired total width W.

The middle section 96 between the end sections 92, 94 may have one or more groups of openings, such as the group of openings 110 shown in FIG. 9, formed in a modified zone 111 of the middle section 96 that are configured to adjust the mechanical properties of the tissue support 90 when implanted, similar to the previously described tissue support 20. It should be appreciated that while the modified zone 111 appears in FIGS. 9-10 to be a visually discrete region of the middle section 96, in some embodiments the modified zone 111 may be visually indistinguishable from the rest of the middle section 96 and/or only visually distinguishable due to the presence of openings or other physical alterations. In some exemplary embodiments, the openings 110 may comprise perforations, indentations, slits, holes, dimples, and/or any other suitable openings to improve gradient mechanical properties of the tissue support 90. In some exemplary embodiments, the tissue support 90 may comprise varying regions that may include one or more localized openings that vary in size, shape, number, spacing, and/or orientation relative to the tissue support 90. In one exemplary embodiment, the openings 110 may be used to alter the mechanical properties selectively across the ATM of the tissue support 90. As previously described, the openings 110 may be disposed parallel or perpendicular to an expected direction of elongation upon implantation.

As can be seen, the openings 110 can be formed identically to one another and each define an opening length OL that is greater than an opening width OW. It should be appreciated that the opening length OL and the opening width OW may be altered as desired. In some exemplary embodiments, the opening length OL and the opening width OW may each correspond to the same percentage of the total length L and the total width W, respectively, i.e., the opening length OL of each opening 110 may be X % of the total length L and the opening width OW of each opening 110 may be X % of the total width W, with X being the same for both the opening length OL and the opening width OW. In other exemplary embodiments, the opening length OL of each opening 110 may be X % of the total length L and the opening width OW of each opening 110 may be Y % of the total width W, with X and Y being different. It should therefore be appreciated that the dimensions of the openings 110 can be adjusted, as desired, to impart the desired mechanical properties to the tissue support 90 following implantation, e.g., elongation characteristics, stiffness, etc.

As shown in FIG. 9, the tissue support 90 may define a width center line 120 extending parallel to the direction of the total width W of the tissue support 90, with the tissue support 90 being mirrored across the width center line 120, i.e., the tissue support 90 is generally symmetrical. While the tissue support 90 is shown as being symmetrical, in some exemplary embodiments the tissue support 90 may be asymmetrical.

With further reference to FIG. 9, and referring now to FIG. 10 as well, the tissue support 90, when shaped and configured for use in a neck lift surgery, may define a first width side 130, which may be referred to as a “chin side,” and a second width side 132, which may be referred to as a “neck side.” The neck side 132, as its name suggests, may be configured for attachment to tissue in the neck of a patient. The chin side 130, on the other hand, may be configured for attachment to tissue adjacent to or on the chin of a patient.

Due to the relative difference in mechanical behavior between the chin and the neck, especially with regards to the amount of relative tissue movement near the chin and/or jawline, a user may decide that the tissue support 90 should have greater flexibility, i.e., ability to stretch, on the chin side 130 of the tissue support 90 compared to the neck side 132 in order to account for the increased amount of natural tissue movement that generally occurs near the chin due to, for example, facial movement. To impart additional flexibility to the chin side 130 of the tissue support 90, but not the neck side 132, the user may elect to form one or more additional cutouts 134, 136 in the chin side 130 of the tissue support 90 but not in the neck side 132; it should be appreciated that the shapes of additional cutouts 134, 136 are illustrated as dashed lines in FIG. 9, with the additional cutouts 134, 136 illustrated as being formed in the tissue support 90 in FIG. 10. A user may decide to form the additional cutouts 134, 136 based on mechanical modeling of the tissues in the chin and neck area, as previously described, and/or based on the user's intuition.

As can be seen, the additional cutouts 134, 136 can be formed to have a generally curved shape that each extend approximately 15-20% of the total length L in a direction of the length L on either side of the width center line 120. Further, the additional cutouts 134, 136 can be formed to each define a respective innermost point 138, 140 that does not reach a length center line 142 of the tissue support 90 extending in the direction of the length L, i.e., the additional cutouts 134, 136 do not extend to the length center line 142 of the tissue support 90. In some exemplary embodiments, the tissue support 90 can have a chin attachment region 137 bounded by the additional cutouts 134, 136 and the modified zone 111, with the chin attachment region 137 being of a sufficient thickness to permit attachment to tissues in the chin region of a patient during a surgical procedure. In some exemplary embodiments, the chin attachment region 137 can have a chin region width CRW defined between the additional cutouts 134, 136 of approximately 2 cm to 5 cm, such as between 2 cm and 3 cm, which allows sufficient material in the chin attachment region 137 to be attached to the chin area in order to maintain attachment of the tissue support 90 in the chin area.

By forming the additional cutouts 134, 136 in the tissue support 90 as shown, the mechanical properties of the tissue support 90 can be well-suited for implantation at the chin and neck areas, with the chin side 130 of the tissue support 90 having greater flexibility to match the natural tissue movement adjacent the chin and neck areas while the neck side 132 of the tissue support 90 has less flexibility to match the relatively lower amount of tissue movement further down the neck area toward the abdomen, as illustrated by respective stretch arrows 144, 146 on each side 130, 132 of the tissue support 90 in FIG. 10. The tissue support 90 can thus support native tissues in a way that avoids inhibiting natural movement of the tissues in regions that naturally have significant and/or frequent movement, i.e., the chin area, while providing increased support for tissues in regions that may not naturally have significant and/or frequent movement and/or require increased support due to more significant atrophy of supportive tissues, i.e., the neck area. In other words, the tissue support 90 can provide sufficient support in areas where significant support of the tissues may be needed, such as in the neck, while not being overly supportive in areas where such a high degree of stiffness of the tissue support 90 would inhibit natural tissue movement and create unnatural tissue movements and/or stiffness, such as the chin and jawline area.

Alternatively or in addition, the middle section 96 of the tissue support 90 may have different mechanical properties than either end section 92, 94 due to enzymatic and/or chemical treatment to, for example, soften the tissue or cross-link the tissue to increase the tissue's stiffness, or affect other properties. It should be appreciated that the chemical and/or enzymatic treatment of the middle section 96 can be combined with material addition to or removal from the middle section 96 to alter the mechanical properties of the tissue support 90. It should therefore be appreciated that the middle section 96 of the tissue support 90 can be configured in many different ways to adjust the mechanical behavior of the tissue support 90 when implanted, e.g., by physically, chemically, and/or enzymatically altering the ATM sheet forming the tissue support 90.

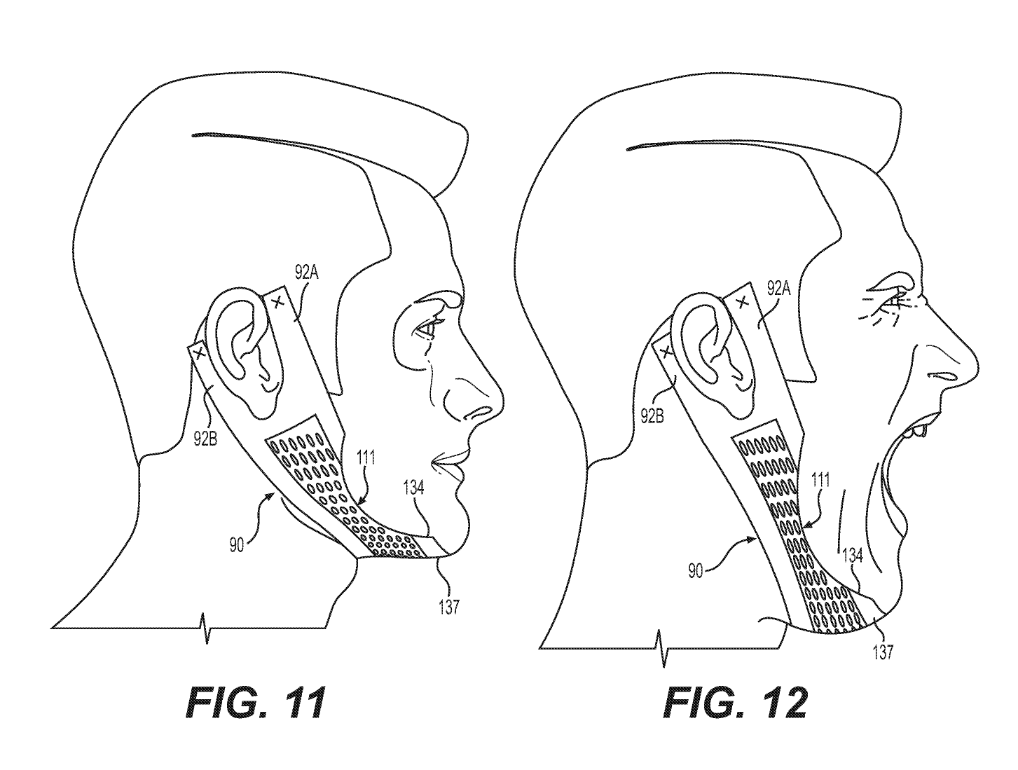

To implant the tissue support 90 shown in FIGS. 9-10, and referring to FIGS. 11-12 as well, a user may first measure a desired overall length of the tissue support 90 and trim part of the first end section 92 and/or second end section 94 so the tissue support 90 has a total length L that the user desires. The user may also remove additional material from the tissue support 90, such as the additional cutouts 134, 136, to adjust the overall mechanical properties of the tissue support 90 for the procedure; in some exemplary embodiments, removal of additional material from the tissue support 90 may be based on mechanical modeling of tissue in the face and/or neck regions of the patient. Before implantation of the tissue support 90, the user forms one or more incisions at desired attachment sites of the patient and can then attach the first end section 92, second end section 94, chin attachment region 137, and any other desired portion of the tissue support 90 to various tissue structures of the patient. In one exemplary embodiment, the user may attach the zygomatic attachment flaps 92A, 94A of the respective end sections 92, 94 to the zygomatic processes of the patient (or to adjacent tissues) and attach the mastoid attachment flaps 92B, 94B to the mastoid processes of the patient (or to adjacent tissues) using one or more anchoring elements, such as sutures. Exemplary attachment points, which may represent suture sites, are illustrated in FIGS. 10-12 as X's on the tissue support 90, and are not limited to just the zygomatic and mastoid processes; as can be seen in FIG. 10, attachment points X may also be in the chin attachment region 137 to attach the tissue support 90 to tissue in the chin area, as shown in FIGS. 11-12, and in attachment zones 148 outside the modified zone 111 in the middle section 96. In some exemplary embodiments, the end sections 92, 94 may be attached to analogous tissue structures on opposite lateral sides of the face and neck. It should be appreciated that attachment of the tissue support 90 to the patient may occur before, simultaneously with, or after the user has performed other aspects of the surgical procedure, such as liposuction or skin removal. After implantation, the user may close the incision with the tissue support 90 in place, with or without performing additional surgical steps, so the tensioned tissue support 90 supports tissues and muscles in contact with the tensioned tissue support 90.

As can be seen in comparing FIGS. 11 and 12, when a patient's face experiences significant movement such as, for example, while yawning, the flexibility of the tissue support 90 adjacent to the chin area allows a more natural movement of supported tissue. The tissue support 90 can thus allow for a lift procedure, such as a neck lift, to produce improved aesthetic appearance while preserving natural tissue movements due to the flexibility in the support 90.

While the tissue products (e.g., acellular dermal matrices), and related methods of treatment, of the present disclosure are described with reference to facial, jawline and neck area reconstructive surgical procedures, it should be understood that the tissue products may be used in abdominal surgical procedures, or any other suitable medical procedure where tissue support products having gradient mechanical properties and/or compliance requirements may be desirable or necessary.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. Accordingly, the invention is not to be considered as limited by the foregoing description.

What is claimed is:

1. A support, comprising:
a sheet of acellular tissue matrix, wherein the sheet of acellular tissue matrix comprises a first end section, a second end section, and a middle section connecting the first and second end sections, wherein at least one group of openings are disposed along at least a portion of the middle section, and wherein the first and second end sections are configured to receive anchoring elements to retain the sheet to at least one of native facial tissue and native neck tissue.

2. The support of claim 1, wherein the sheet of acellular tissue matrix comprises a dermal tissue matrix.

3. The support of claim 1, wherein the acellular tissue matrix is a porcine tissue matrix.

4. The support of claim 1, wherein the acellular tissue matrix is a human tissue matrix.

5. The support of claim 1, wherein at least one of the first end section or the second end section has a first attachment flap and a second attachment flap separated by a cutout.

6. The support of claim 1, wherein the sheet defines a first width side and a second width side, the sheet having at least one cutout formed in the second width side.

7. The support of claim 6, wherein the first width side of the sheet does not have any cutouts formed therein.

8. The support of claim 6, wherein the at least one cutout does not extend to a length center line of the sheet.

9. The support of claim 1, wherein each opening from the group of openings is uniform in shape and size.

10. The support of claim 1, wherein the anchoring elements comprise at least one of sutures, barbed sutures, suture bites, clips, meshes, or biological adhesives.

11. A facial treatment device, comprising:
a tissue support containing an acellular tissue matrix,
wherein a group of perforations are disposed along at least a portion of a modified zone of the tissue support.

12. The device of claim 11, wherein the acellular tissue matrix is a porcine tissue matrix.

13. The device of claim 11, wherein the acellular tissue matrix is a human tissue matrix.

14. The device of claim 11, wherein the tissue support further comprises two anchoring sections disposed on opposite ends of the tissue support.

15. The device of claim 14, wherein the anchoring sections further include anchoring elements to retain the tissue support member to at least one of native facial tissue and neck tissue.

16. The device of claim 15, wherein the anchoring elements comprise at least one of sutures, barbed sutures, suture bites, clips, meshes, or biological adhesives.

17. A method of treatment, comprising:
implanting a device comprising a support according to claim 1 within a cranio-facial structure and a neck structure.

18. The method of claim 17, wherein the method comprises anchoring at least one of the first or second sections to tissue within a cranio-facial structure and a neck structure.

19. The method of claim 18, wherein the method comprises anchoring the first section to a first tissue structure and the second section to an analogous second tissue structure that is laterally opposite the first tissue structure to provide support to tissue.
